(19) United States
(12) Patent Application Publication
(10) Pub. No.: US 2007/0167868 A1
(43) Pub. Date: Jul. 19, 2007

(54) Ergonomic needle tissue harvesting instrument not requiring a stylet

(75) Inventor: Jude S. Sauer, Rochester, NY (US)

Correspondence Address: Stephen B. Salai, Esq.;Harter, Secrest & Emery LLP, 1600 Bausch & Lomb Place, Rochester, NY, 14604-2711, US

(73) Assignee: LSI Solutions, Inc., Victor, NY (US)

(21) Appl. No.: 11/334,314

(22) Filed: Jan. 18, 2006

(51) Int. Cl. A61B 10/00

(52) U.S. Cl. 600/564; 600/562

(57) ABSTRACT

A medical diagnostic instrument for the ergonomic effective and safe harvesting of specimen at targeted remote tissue sites includes a pistol grip style handle with a hand activated lever and a specialized elongated flexible double tube needle shaft inside of a protective sheath for use, in a preferred embodiment, within an endoscope. The length of needle shaft is adjusted using locking buttons. Moving another set of buttons sets the needle penetration depth. With a squeeze of the lever, a novel thin band drive mechanism advances the needle shaft out of its sheath, through adjacent tissue and into the targeted site. The double tube needle shaft incorporates a pointed distal tip attached to an innermost “grater” needle, which has sharp edged tissue cutting holes or “grating” features that are exposed by retracting back an overlying cover tube. Sliding back the cover tube is achieved via a cam. The sharp cutting surfaces of the grater tube communicate directly with a vacuum source attached near the handle. Oscillation of the vacuum augmented grater tube back and forth within the targeted tissue yields small pieces of tissue for harvest. When adequate amounts of tissue are drawn into the grater tube, the cover tube is re-advanced over the distal grater tube, the needle shaft is retracted back into the sheath and the instrument along with the harvested specimen are removed from the patient.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention Field of the Invention

This invention relates generally to a needle tissue harvesting instrument and more particularly to such a device including a needle having one or more tissue scraping apertures in a side wall of the needle and a cover tube coaxially disposed on the needle and slidably movable from an extended position covering the needle to a retracted position exposing one or more of the tissue scraping apertures.

2. Description of Related Art

Diagnostic patient interventions are used to learn more about a patient's physical condition. Non-invasive diagnostics techniques (e.g., taking a patient's temperature, radiographic imaging, electrocardiographic monitoring, etc.) alone often do not provide sufficient data to establish important diagnoses; directly obtaining actual tissues samples for pathologic evaluation is routinely necessary to confirm or rule out critical diagnoses, such as the presence of cancerous cells. The size range for harvested tissue varies according to the patient's specific conditions. Physicians' can obtain larger tissue structures (e.g., a gross surgical specimen, such as an entire organ), smaller samples that have some intact tissue architecture (e.g., a core biopsy specimen for microscopic histologic evaluation) or cellular sized tissue fragments (e.g., fine needle aspirations for cytologic evaluation). To provide better patient outcomes, improved technologies are needed to continue to reduce the invasiveness and potential morbidity of harvesting representative, excellent quality, patient tissue samples.

Efforts to improve a physician's ability to see more than just the outer characteristics of a patient are essential to modern health care. For centuries, health care practitioners could only use their direct vision to view their patient's body surfaces, exposed orifices or anatomy exposed through open incisions or wounds. The use of radiographic techniques (e.g., X-ray, CT and MRI) and endoscopic techniques (e.g., colonoscopy, gastroscopy and laparoscopy), along with combinations of these modalities, now routinely provides clinically significant data. An endoscopic technique for viewing internal patient body cavities was first reported in 1805. Important advancements in endoscopic techniques (e.g., laparoscopic surgery in 1901, flexible fiber optic endoscopes in 1957, endoscopic retrograde cholangiopancreatography (E.R.C.P) in 1968, laparoscopic cholecystectomy in 1988, etc.) helped usher in this era of modern medicine. Improvements to endoscopic technology, including cooler light sources, flexible and steerable endoscopes, endoscopes with attached working channels for passing instruments, and the use of video image acquisition technology, improved patient viewing techniques, which yielded significant improvements in appropriate therapeutics.

In the late 1980's, ultrasonic transducers were added to the ends of endoscopes to launch the age of Endoscopic Ultrasound (usually abbreviated, EUS). Medical ultrasound devices had already been used to obtain externally accessible images (e.g., for breast screening or fetal evaluation). EUS provides electronic image processing to enable endoscopists to enhance clinical diagnoses by viewing ultrasonic images of tissue structures under the surface. The advent of linear array EUS facilitated the use of ultrasound guided fine needle aspiration biopsy (EUS-FNA) initially for gastrointestinal diseases. In 1992, Dr. P. Vilmann and colleagues pioneered the transformation of using EUS from only looking at tissue structures to harvesting tissue samples for cytological analysis (Gastrointestinal Endosc. 1992:38:172-3). By obtaining actual pieces of pancreas tissue to examine under a microscope, more definitive diagnoses were possible along with the improved ability for accessing prognosis and best therapeutic interventions. Their device incorporated a narrow gauge needle to penetrate through native bowel and into the imaged pancreatic harvest site. The intent was to obtain an adequate tissue sample, while causing as little trauma as possible to the non-pathologic tissue and thereby minimizing the risk of clinical leakage of the bowel contents through the needle tract, bleeding caused by perforating more vascular structures, fistula formation, etc. Expansion of use of this approach to other body areas, such as the mediastinum (i.e., the area around the heart), represents further opportunity to use FNA to help a broader population of patients.

While the first EUS-FNA devices proved reasonably effective, very little functional improvement in design has occurred since. All commercially available EUS-FNA devices incorporate three interlocking components: a cylindrical handle assembly, a long needle and a wire stylet. The relatively unchanged original design resembles a syringe with a plunger attached to a protective sheath containing a long, flexible needle with a coaxial thin wire (the stylet) inside the needle. The sheath guides the needle through the channel of the endoscope. The plastic or metal tube-like handle of the handle assembly includes a needle piston or plunger, which controls excursions of the needle in and out of the sheath, and attaches to a flexible sheath. The continuous needle courses through the handle and sheath. By passing all the way though the device from the top of the handle to just beyond the needle tip, the removable stylet is intended to occlude the needle tip during initial tissue penetration to prevent non-targeted tissue from being inadvertently harvested. The ungainly stylet, which is long and extremely thin, needs to be removed entirely from the device to allow aspiration of tissue into the needle. To reuse this type of device in the same patient, as is frequently necessary in EUS-FNA procedures, the stylet must be successfully reinserted back through the now contaminated device. This is difficult, or at least time consuming. Commercially available current EUS-FNA product designs are distinguishable mostly regarding packaging or assembly considerations and whether components from these products are intended for re-sterilization and subsequent use with more than one patient (i.e., re-useable) verses those provided already sterilized for single patient use (i.e., disposable).

The first widely utilized EUS-FNA device was marketed by Medi-Globe® (Medi-Globe Corporation, Tempe, Ariz., www.gip-med.de) was a re-useable metal handled assembly with single patient use needle and stylet. Olympus® (Olympus Medical Systems Corporation, Tokyo, Japan) now also sells a re-useable handle device, like the Medi-Globe® product, with a disposable needle and stylet. Cook® (Wilson-Cook Medical GI Endoscopy, Winston-Salem, N.C.) and Con-Med® (Endoscopic Technologies, Billerica, Mass.) offer completely disposable EUS-FNA device products currently marketed ECHOTIP® Ultra Endoscopic Ultrasound Needle and Vizeon™, respectively. To date, design improvements to these known products seem to be limited to alternative bevels on needle tips, a variety of stylet tips, more kink resistant stylet materials (e.g., nitinol instead of just stainless steel), needle size offerings (now typically 19, 22 and 28 gauge), tip surfaces alterations for improved echogenicity, more flexible and puncture resistant sheath styles, extendable sheath connectors to accommodate a variety of endoscope working channel lengths, etc. Claims of improved ergonomics in these available products appear to be limited to attempts to improve how these products are assembled in the field prior to patient use or modest stylistic changes to the shape, materials or surface texture of otherwise cylindrical syringe-like handles.

Deployment and oscillation of the currently available EUS-FNA device needle to obtain a tissue sample usually requires gripping the plunger component with two fingers on one hand and moving the plunger repeatedly up and down relative to the syringe housing. This non-ergonomic needle oscillation technique, sometimes called the “dart technique,” remains very awkward, fatiguing and can shake the attached gastroscope so much that it loses its position to image the targeted remote site. Another, perhaps the most problematic shortcoming of the current technology, is the requirement that all existing devices need a removable stylet within the needle. This long (typically over 125 cm) thin (standard outside diameter of 0.018″) metal wire, usually with a pointed distal end, must be appropriately oriented and maintained inside of the FNA needle to minimize the risk contamination of the harvested specimen as the needle passes through non-targeted tissue. This unwieldy, cumbersome, dangerous wire is often removed and reinserted multiple times during an EUS-FNA procedure. While inappropriate contact with a stylet can cause patient trauma, stylets pose an even more frequent threat to the endoscopist and endoscopy team from exposure to patient bodily fluids or even puncture wounds to the staffs' skin or eyes.

Existing needle aspiration technology, especially for EUS-FNA techniques, has not been optimized for ergonomics, effectiveness or medical team and patient safety. Better devices for fine needle aspiration can provide better patient care by improving this important diagnostic modality.

### BRIEF SUMMARY OF THE INVENTION

Briefly stated and in accordance with certain presently preferred embodiments of the invention, a medical diagnostic instrument for the ergonomic, effective and safe harvesting of specimen at targeted remote tissue sites includes a pistol grip style handle with a hand activated lever, customized adjustment features and a specialized elongated flexible double tube needle shaft inside of a protective sheath.

In accordance with another aspect of the invention, the instrument is attached to a port in the proximal end of an echoendoscope with its sheathed flexible needle shaft placed within the scope's working channel. The length of needle shaft positioned inside the echoendoscope is adjusted using the button mediated needle shaft length adjustment feature. After imaging the location of the targeted lesion, the needle penetration depth is set by moving another set of buttons. With a squeeze of the lever, a novel thin band drive mechanism advances the needle shaft out of its sheath, away from the distal tip of the scope, through adjacent tissue and into the targeted site. The double tube needle shaft incorporates a pointed distal tip attached to the innermost tube, called the “grater” tube. The distal grater tube integrates sharp edged tissue cutting or “grating” features that are exposed by retracting back a overlying cover tube. A cam mechanism located near the handle is provided to hold and then slide the cover tube into the mid-open or max-open or positions back to the closed position. The now exposed sharp cutting surfaces of the grater tube communicate directly through the grater tube with a vacuum source attached near the handle. Oscillation of the vacuum pressurized (augmented) grater tube back and forth within the targeted tissue yields small pieces of tissue for harvest. When adequate amounts of tissue are drawn into the grater tube, the cover tube is re-advanced over the distal grater tube, the needle shaft is retracted back into the sheath and the instrument along with the harvested specimen are removed from the patient

In accordance with another aspect of the invention, a similar ergonomic device that enables enhancement of obtaining larger core biopsy tissue samples.

In accordance with another aspect of the invention, a similar ergonomic device that enables enhancement of obtaining tissue samples using a traditional needle or needle with indwelling stylet.

### BRIEF DESCRIPTION OF THE SEVERAL VIEWS OF THE DRAWING(S)

The foregoing objects, features and advantages of the invention will become more apparent from a reading of the following description in connection with the accompanying drawings, in which:

FIG. 1 is a perspective view of the fine needle aspiration instrument in accordance with the present invention;

FIG. 2 is a perspective view of the fine needle aspiration instrument of FIG. 1 shown attached to an echoendoscope;

FIG. 3 is a perspective view of the fine needle aspiration instrument of FIG. 1 in which the right cover of the housing of the instrument is removed;

FIG. 4A is a partially exploded perspective view of the fine needle aspiration instrument of FIG. 1 in which the handle halves are separated;

FIG. 4B is an exploded perspective view of the fine needle aspiration instrument of FIG. 1;

FIG. 4C is an exploded perspective view of select components of the fine needle aspiration instrument of FIG. 1;

FIG. 5A is an exploded perspective view of the shaft length adjustment subassembly of FIG. 4;

FIG. 5B is a perspective view of the shaft length adjustment subassembly of FIG. 4 with the top half of the scope mount cut away to show the articulation at the faceted universal joint between the distal shaft length adjuster tube and the proximal scope mount;

FIGS. 6A and 6B are partially sectioned perspective views from the top right orientation of the spring lock button from the shaft length adjustment components of FIG. 3;

FIG. 7 is a perspective view of an assembled shaft length adjustment subassembly of FIG. 3 now advanced about half way forward;

FIG. 8A is a top perspective view of the fine needle aspiration instrument of FIG. 1;

FIG. 8B is a top perspective view of the fine needle aspiration instrument of FIG. 1 showing the shaft length adjustment buttons about half way forward;

FIG. 8C is a top perspective view of the fine needle aspiration instrument of FIG. 1 showing the shaft length adjustment buttons fully forward;

FIGS. 9A and 9B show right perspective views of the universal joint of the scope mount component of the shaft length adjustment mechanism of FIG. 1 fully flexed in an upward orientation and to the right orientation, respectively;

FIG. 10 is a close-up partially sectioned perspective view of the back of the fine needle aspiration instrument of FIG. 3;

FIG. 11A is a top perspective view of the fine needle aspiration instrument of FIG. 1 showing needle control tube and spring button fully back;

FIG. 11B is a close-up schematic view of the needle shaft assembly outlined inside of the distal flexible sheath of the fine needle aspiration instrument of FIG. 11A;

FIG. 11C is a top perspective view of the fine needle aspiration instrument of FIG. 1 showing the needle control button now advanced fully forward;

FIG. 11D is a close-up schematic view of the needle shaft assembly outlined inside of the distal flexible sheath of the fine needle aspiration instrument of FIG. 11C;

FIG. 11E is a top perspective view of the fine needle aspiration instrument of FIG. 1 showing the needle control button still fully forward, but the needle control tube is advanced about half way forward along with its attached needle assembly, the distal tip of which now extends out beyond the distal end of the flexible sheath;

FIG. 11F is a close-up schematic view of the needle shaft now extending beyond the distal end of the flexible sheath of the fine needle aspiration instrument of FIG. 11 ;

FIG. 11G is a top perspective view of the fine needle aspiration instrument of FIG. 1 showing the needle control button, the needle control tube and attached needle assembly all fully forward with the distal needle tip now fully extended beyond the distal end of the flexible sheath;

FIG. 11H is a close-up schematic view of the needle shaft now fully extended beyond the distal end of the flexible sheath of the fine needle aspiration instrument of FIG. 11G;

FIG. 12 is a perspective view from a bottom viewing orientation of selected components of the fine needle aspiration instrument of FIG. 3 in which the right cover of the housing of the instrument and the spring lock buttons and the shaft length adjuster tube are removed;

FIG. 13A is an exploded perspective view of selected components from FIG. 12 showing a section of the slotted cover tube, the drive tang connected to the grater tube and the top section of the drive band;

FIG. 13B is an exploded perspective view of selected components of FIG. 13A showing a section of the slotted cover tube covering a section of the grater tube with its connected drive tang directed toward the receiving slot in the top section of the drive band;

FIG. 14A is a perspective view from a bottom viewing orientation of selected components of the fine needle aspiration instrument of FIG. 12 showing the needle assembly fully retracted;

FIG. 14B is a perspective close up view of the call out from FIG. 14A highlighting the relative orientation of the band drive connection to the drive tang, grater tube and cover tube;

FIG. 14C is a perspective view from a bottom viewing orientation of selected components of the fine needle aspiration instrument of FIG. 12 showing the partially advanced needle assembly;

FIG. 14D is a perspective view from a bottom viewing orientation of selected components of the fine needle aspiration instrument of FIG. 12 showing the fully advanced needle;

FIG. 15A is a perspective view of the distal needle assembly of FIG. 11 with the cover tube pulled slightly back to expose the most distal sharpened hole in the grater tube;

FIG. 15B is an enlarged section view of FIG. 15A highlighting the edges of the sharpened hole of grater tube and the needle tip;

FIG. 15C provides a perspective view of the distal tip of an alternative embodiment to aspiration needle shown in 15A with a larger single hole and sharpened distal tube for core biopsy;

FIG. 15D provides a perspective view of the distal tip of a traditional fine needle aspiration needle shown with a beveled stylet wire in place in the needle's lumen;

FIG. 16A is a perspective view of the fine needle aspiration instrument of FIG. 1 shown with the pivoting lever fully back and the needle cam fully forward in its fully closed position thereby holding the cover tube fully forward over the distal grater tube; note the circles calling out the needle cam area and the distal needle;

FIG. 16B is a perspective close up view of the single circle call out from FIG. 16A with the right needle cam section removed and a cut out in the underlying needle control tube revealing the cam follower in its fully forward position;

FIG. 16C is a perspective close up view of the double circle call out from FIG. 16A showing the cover tube fully forward and in contact with the needle tip;

FIG. 16D is a perspective view of the fine needle aspiration instrument of FIG. 1 shown with the pivoting lever fully back and the needle cam oriented upward in its mid open position thereby drawing the cover tube half way back over the distal grater tube; note the circles calling out the needle cam area and the distal needle;

FIG. 16E is a perspective close up view of the single circle call out from FIG. 16D shown with the right needle cam section removed and a cut out in the underlying needle control tube revealing the cam follower in its half way back position;

FIG. 16F is a perspective close up view of the double circle call out from FIG. 16D showing the cover tube half way retracted exposing half of the sharpened holes in the distal grater tube;

FIG. 16G is a perspective view of the fine needle aspiration instrument of FIG. 1 showing the pivoting lever fully back and the needle cam oriented fully back in its max open position thereby drawing the cover tube fully back over the distal grater tube; note the circles calling out the needle cam area and the distal needle;

FIG. 16H is a perspective close up view of the single circle call out from FIG. 16G with the right needle cam section removed and a cut out in the underlying needle control tube revealing the cam follower in its fully back position and moving the attached proximal cover tube to its maximum retraction position;

FIG. 16J is a perspective close up view of the double circle call out from FIG. 16G showing the cover tube fully retracted exposing all of the sharpened holes in the distal grater tube;

FIG. 17A is a schematic representation of the distal end of an echoendoscope with a protruding distal end of a flexible sheath positioned over a tissue segment containing an imaged lesion;

FIG. 17B is a schematic representation of the distal end of an echoendoscope with a protruding distal end of a flexible sheath positioned over a tissue segment containing an imaged lesion with a non-retracted cover tube penetrating through the overlying tissue and deeply into the lesion;

FIG. 17C is a schematic representation of the distal end of an echoendoscope with a protruding distal end of a flexible sheath positioned over a tissue segment containing an imaged lesion, the needle penetrating through the overlying tissue deeply into the lesion, and the cover tube fully retracted back to expose the sharpened holes of the grater tube;

FIG. 17D is a schematic representation of the distal end of an echoendoscope with a protruding distal end of a flexible sheath positioned over a tissue segment containing an imaged lesion, the needle with the cover tube fully retracted exposing the sharpened holes of the grater tube now pulled partially back up through the lesion;

FIG. 17E is a schematic representation of the distal end of an echoendoscope with a protruding distal end of a flexible sheath positioned over a tissue segment containing an imaged lesion at completion of the oscillation harvesting technique and the double tube needle now with the cover tube fully forward covering the sharpened holes of the grater tube;

FIG. 17F is a schematic representation of the distal end of an echoendoscope with a protruding distal end of a flexible sheath positioned over a tissue segment containing an imaged and also showing a representative needle track through the overlying tissue and the specimen harvest site in the imaged lesion;

### DETAILED DESCRIPTION OF THE INVENTION

In accordance with a presently preferred embodiment of the invention, a medical diagnostic instrument is provided for the ergonomic, effective and safe harvesting of specimen at targeted remote tissue sites. The instrument includes a pistol grip style handle with a hand activated lever, customized adjustment features and a specialized elongated flexible double tube needle shaft inside of a protective sheath. For clarity, these novel design features will be presented here in the sequence that they are typically encountered in a routine EUS-FNA procedure. The instrument is attached to port in the proximal end of an echoendoscope with its sheathed flexible needle shaft placed within the scope's working channel. The length of needle shaft positioned inside the echoendoscope is adjusted using the button mediated needle shaft length adjustment feature. After imaging the location of the targeted lesion, the needle penetration depth is set by moving another set of buttons. With a squeeze of the lever, a novel thin band drive mechanism advances the needle shaft out of its sheath, away from the distal tip of the scope, through adjacent tissue and into the targeted site. The double tube needle shaft incorporates a pointed distal tip attached to the innermost tube, called the “grater” tube. The distal grater tube integrates sharp edged tissue cutting or “grating” features that are exposed by retracting back a overlying cover tube. Sliding back the cover tube is achieved via a cam mechanism located near the handle. The now exposed sharp cutting surfaces of the grater tube communicate directly through the grater tube with a vacuum source attached near the handle. Oscillation of the vacuum pressurized (augmented) grater tube back and forth within the targeted tissue yields small pieces of tissue for harvest. When adequate amounts of tissue are drawn into the grater tube, the cover tube is re-advanced over the distal grater tube, the needle shaft is retracted back into the sheath and the instrument along with the harvested specimen are removed from the patient.

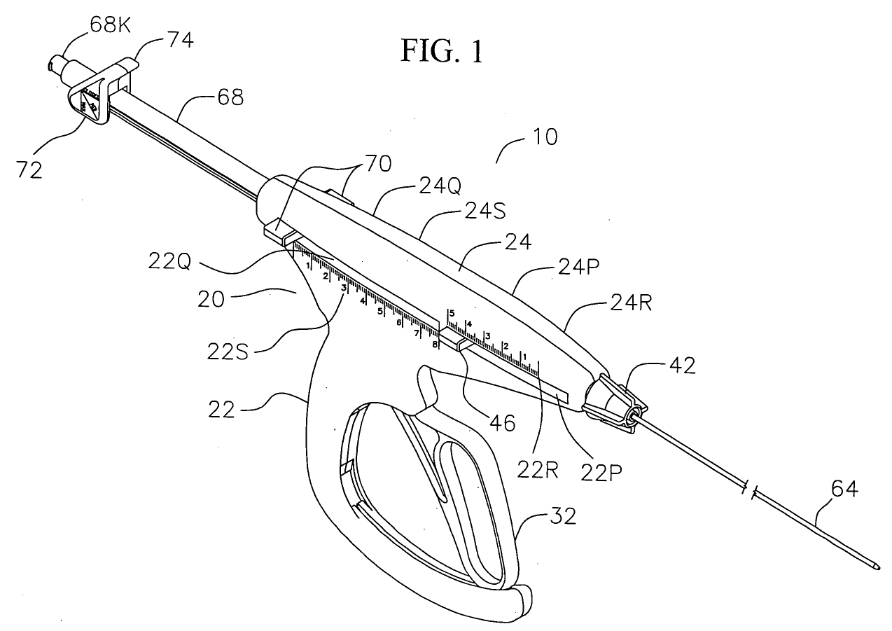

Referring to FIG. 1, a perspective view of the fine needle aspiration instrument 10, is shown, A handle assembly 20 is constructed from a right handle portion 22 and a left handle portion 24 which are constructed of an injection molded plastic or the like and to which subsequent components are attached.

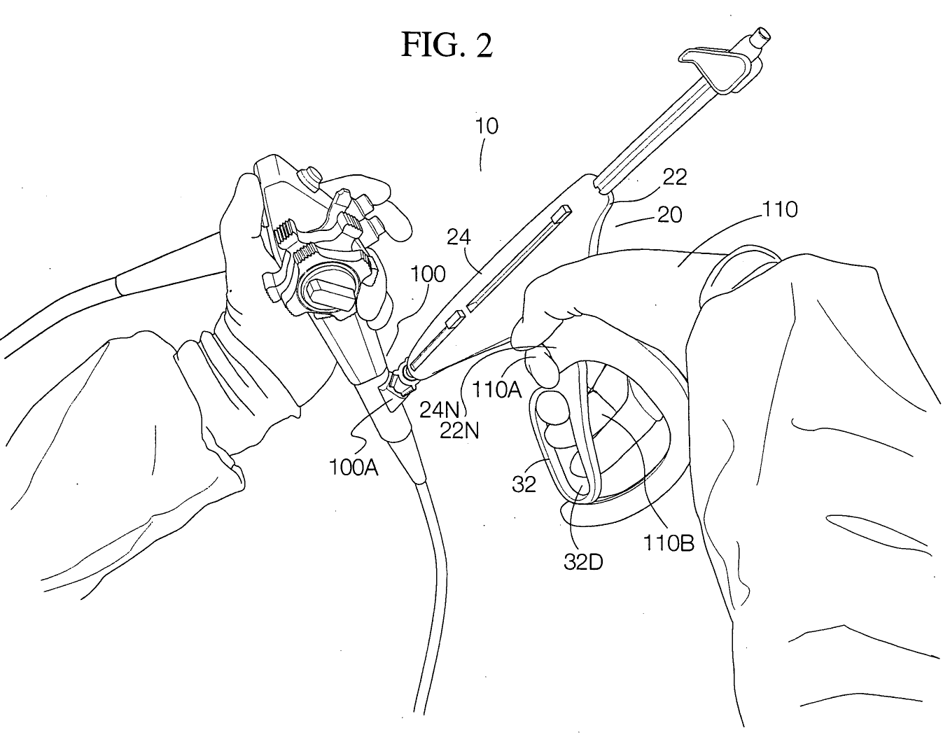

A lever 32 provides for oscillatory operation of internal components of the instrument as described in later figures. Distally, a flex sheath 64 protrudes from a scope mount 42. The scope mount 42 engages an echoendoscope 100 shown in FIG. 2 and described in additional detail in FIGS. 5A and 5B. Additionally, a means to accurately gauge the deployment of later described features is provided by a shaft adjustment scale 22R and 24R (hidden from view) and a needle control adjustment scale 22S and 24S (also hidden from view) which outline a shaft adjustment button slot 22P and 24P (hidden from view) providing a linear guide for a spring lock button 46 and a needle control button slot 22Q and 24Q (also hidden from view) providing a means for linearly guiding a spring lock button 70, respectively. Protruding from the proximal end of the fine needle aspiration instrument 10 is a needle control tube or plunger 68 and an incorporated female luer lock fitting 68K to which a right cam half 72 and a left cam half 74 are assembled.

FIG. 2 is a perspective view of the fine needle aspiration instrument 10 shown attached to the proximal of the working channel 110A of an echoendoscope 100. The fine needle aspiration instrument 10 is contoured such that it is comfortably hand held within the palm of the hand 110 meant to wrap around the handle assembly 20 with an index finger 110A placed upon a finger grip 22N and 24N of the right handle portion 22 and left handle portion 24, respectively. Remaining fingers 11 OB are placed within a finger opening 32D of the lever 32 to provide later described oscillatory motion of additional components within the fine needle aspiration instrument 10.

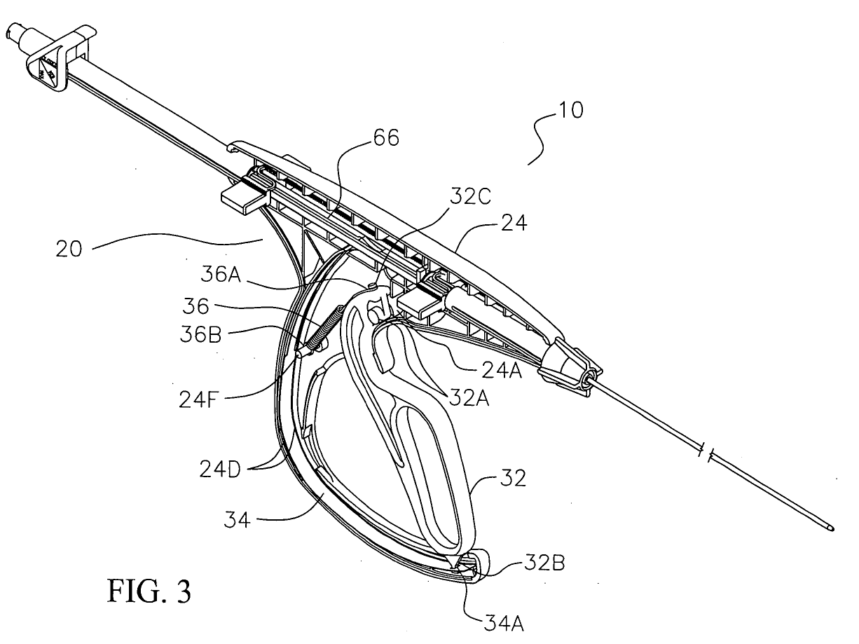

FIG. 3, a perspective view of the fine needle aspiration instrument 10, is shown without the right handle portion 22. The lever 32 is of an injection molded design providing ergonomic function housed with the handle assembly 20 by way of a cylindrical pivot 32A which is circumferentially engaged by a lever pivot 24A of left handle portion 24, and also mirrored as a lever pivot 22A in the right handle portion 22 (not shown). A spring 36 is fixedly attached at one end to a spring post 32C of the lever 32 by way of a hook 36A and at another end to a lever spring post 24F of the left handle portion 24 via an incorporated loop 36B. The spring 36 is of a commercially available spring material such as stainless steel and operates in accordance with common extension spring principles.

A tab 32B at the distal end of lever 32 engages a lever tab opening 34A of a band drive 34 which provides a novel means of directly related lever 32 and band drive 34 actuation. The band drive 34 can be of a commercially available flat material such as stainless steel or plastic. The band drive 34 is engaged by a drive band wall 24D of the left handle portion 24 and mirrored as a drive band wall 22D on the right handle portion 22 (not shown). The drive band wall 24D (and 22D) provide for a means of laterally constraining the drive band 34 within the handle assembly 20 yet allowing free linear motion. The drive band 34 is engaged with a band guide 66 whose functions will be described in later figures.

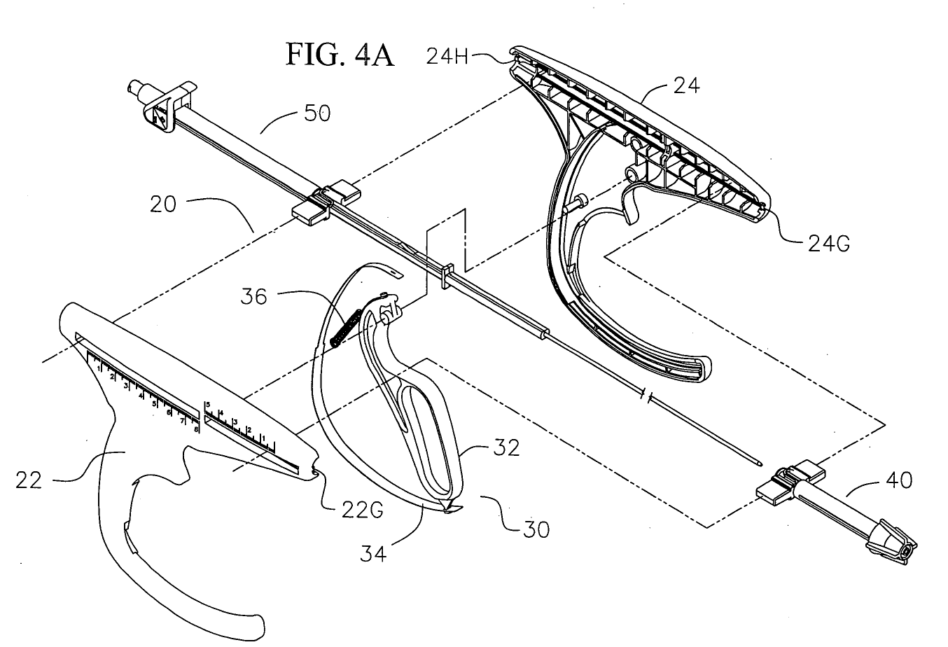

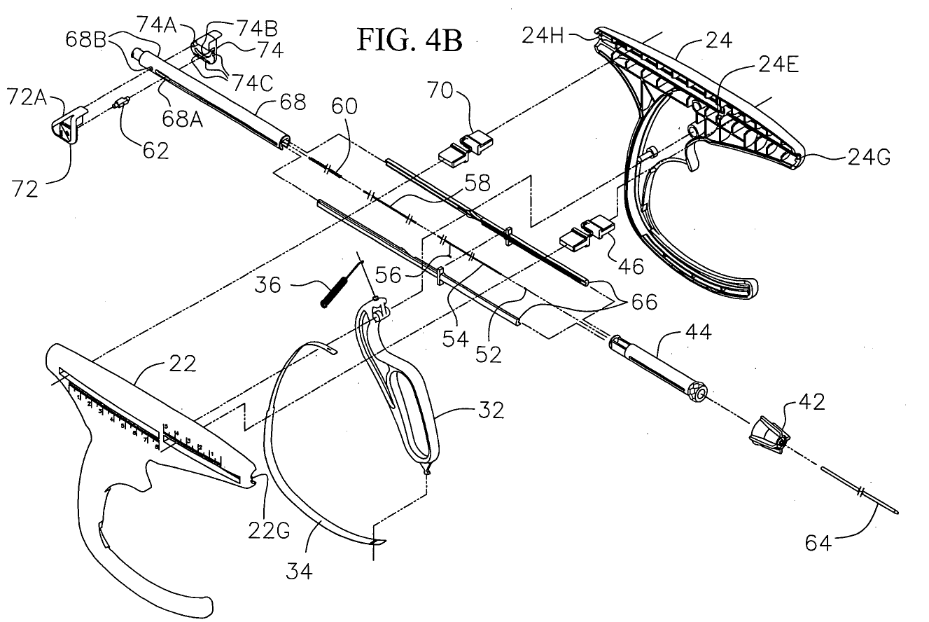

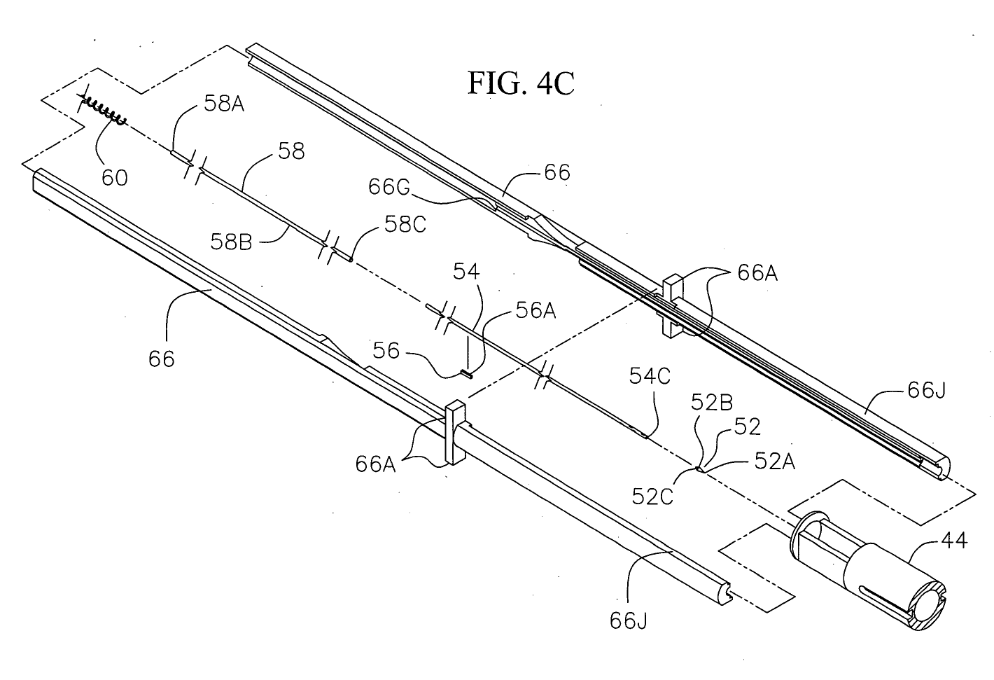

FIGS. 4A through 4C are perspective views of the fine needle aspiration instrument 10 illustrating the assembly structure of this preferred embodiment. FIG. 4A is a partially exploded perspective view of the fine needle aspiration instrument detailing the handle assembly 20, a band drive assembly 30, a shaft length adjuster assembly 40, and a needle control assembly 50. The handle assembly 20 is comprised of the right handle portion 22 and the left handle portion 24. The band drive assembly 30 imparts the lever 32, the band drive 34, and the lever spring 36.

FIG. 4B is a fully exploded perspective view of the fine needle aspiration instrument 10. The right handle portion 22 and left handle portion 24 house the lever 32 and its attached band drive 34 and a lever spring 36. The disassembled shaft length adjuster assembly 40 of FIG. 4A is comprised of a scope mount 42, a shaft adjuster tube 44, and a spring lock button 46. The disassembled needle control assembly 50 is comprised of a needle tip 52, a grater tube 54, a drive tang 56, a cover tube 58, a needle spring 60, a cam follower 62, a flex sheath 64, band guides 66, a needle control tube 68, a spring lock button 70, and a right and left cam half 72 and 74, respectively. The right and left cam halves 72 and 74, respectively, are of injection molded plastic or the like and attach to a cam pivot 68B of the needle control tube via a pivot 72A (not shown) and 74A of the right and left cam half 72 and 74, respectively.

FIG. 4C is an exploded perspective view of select components of the fine needle aspiration instrument 10. The needle tip 52 is permanently attached such that an insert feature 52C mates circumferentially and a shoulder 52B flush to a distal opening 54C of the grater tube 54. The needle tip 52 is preferably made of a stainless steel with a sharp pointed end 52A. The grater tube 54 is also of a stainless steel material and can be similar in nature to commercially available medical tubing. The grater tube 54 and needle tip 52 may be joined by means such as press fitting, or by methods such as welding or brazing. The cover tube 58 longitudinally engages the grater tube 54 such that the needle tip 52 mates with a distal chamfer 58C. The cover tube 58 is preferably made of stainless steel tubing and provides support for the grater tube 54 while allowing linear motion. The drive tang 56 attaches to the grater tube 54 through the drive tang opening 58B of the cover tube 58 via a contoured surface 56A. The drive tang 56 and grater tube 54 are permanently attached via a microwelding process. The interface of the drive tang opening 58B of the cover tube 58 with the fixedly attached drive tang 56 and grater tube 54 provide for a controlled linear motion between the grater tube 54 and the cover tube 58.

The band guide 66 encompasses the above mentioned components and is meant to serve as a stabilizer and as a linear guide for the components. The band guide 66 can be formed of a mirrored injection molded design. The band guide rests within a mounting tab recess 22E (not shown) and 24E of the right handle portion 22 and left handle portion 24 respectively (shown in FIG. 4B) via mounting tabs 66A. The assembled band guide 66 halves are accepted and provide for support of the shaft adjuster tube 44 by way of a shaft adjuster bearing surface 66J. Proximally, a needle spring 60 is assembled concentrically within the band guide 66 and over the cover tube 58 and rests upon a spring seat 66G. The needle spring 60 is of a common compression spring design and can be fashioned from medical grade steel.

FIGS. 5A and 5B are perspective views of the shaft length adjuster assembly 40 of the fine needle aspiration instrument 10. FIG. 5A is an exploded view illustrating the assembly of the shaft length adjuster assembly 40 wherein, distally, the shaft adjuster tube 44 accepts onto a faceted or textured ball 44A a scope mount 42, and proximally a translational constraint bearing surface 46C of the spring lock button 46 is assembled between button translation constraints 44E. A flexure clearance 44F of shaft adjuster tube 44 is provided to allow for a connecting flexure 46F of spring lock button 46 while a bearing surface 44G of shaft adjuster tube 44 allows for interfacing with a contact surface 46B of spring lock button 46. FIG. 5B is a perspective view offering a partial section of the distal end of the shaft length adjuster assembly 40 wherein the scope mount 42 is cut away and a pivot ball socket 42A receiving a textured ball 44A of shaft adjuster tube 44 is visible. The ball and socket joint created by the mating of the shaft adjuster tube 44 and the scope mount 42 is accentuated by the features of the two components. Both the shaft adjuster tube 44 and the scope mount 42 are of a plastic injection molded design and can be textured or faceted to allow for positive, tactile positioning of said components. The scope mount 42 offers a male luer fitting 42B and a female luer fitting 42C thread to accommodate an echoendoscope 100 (best represented in FIG. 2). Additionally, finger tabs 42E are provided to allow for tactile digital manipulation. The spring lock button 46 is a plastic injection molded part offering an innovative means of providing an integrated and repeatedly compressible part with which locking teeth 46E can engage with and disengage from locking teeth of right handle portion 22 and left handle portion 24 of handle assembly 20. An anti-rotation slot 44D is provided for engagement with a shaft adjuster tube anti-rotation nub 22G and 24G of the right handle portion 22 and left handle portion 24, respectively (which are best depicted in FIGS. 4A and 4B).

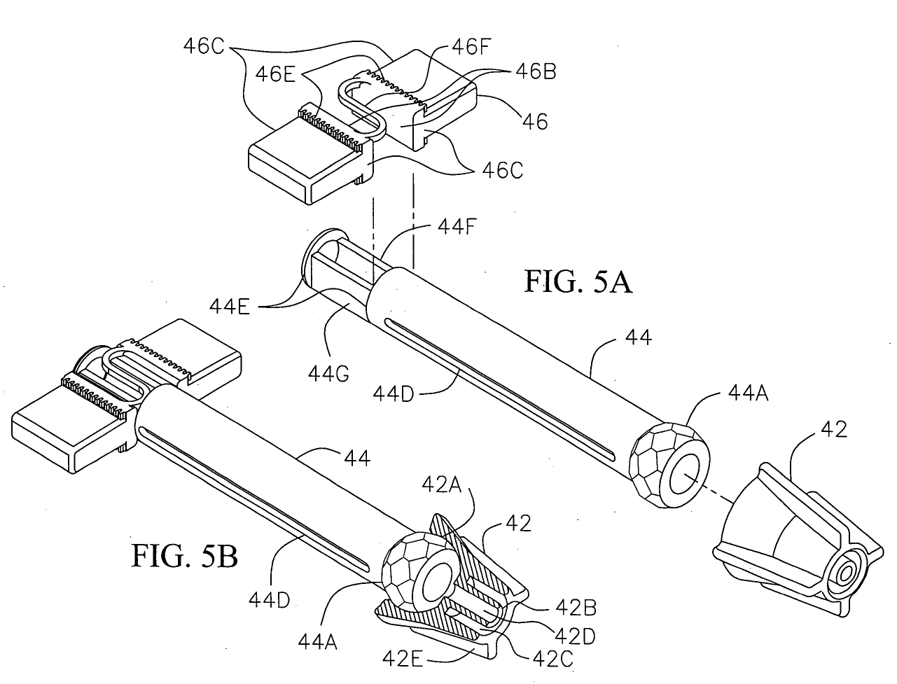

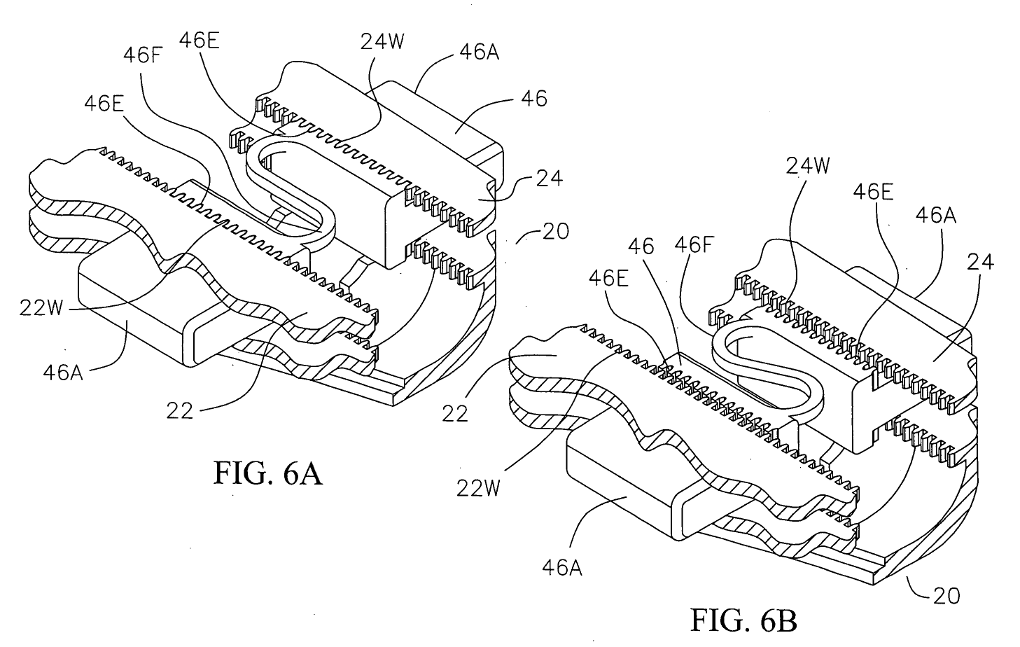

FIGS. 6A and 6B are partially sectioned perspective views of the spring lock button 46 within the handle assembly 20. FIG. 6A illustrates push surfaces 46A of the spring lock button 46 fully apart, the connecting flexure 46F fully expanded, and the locking teeth 46E interlocking with the locking teeth 22W and 24W of the right handle portion 22 and the left handle portion 24, respectively. The connecting flexure 46F keeps the push surfaces 46A aligned in unison during spring button 46 repositioning. FIG. 6B illustrates push surfaces 46A of the spring button 46 compressed, the connecting flexure 46F fully collapsed, and the locking teeth 46E disengaged from the locking teeth 22W and 24W of the right handle portion 22 and the left handle portion 24, respectively.

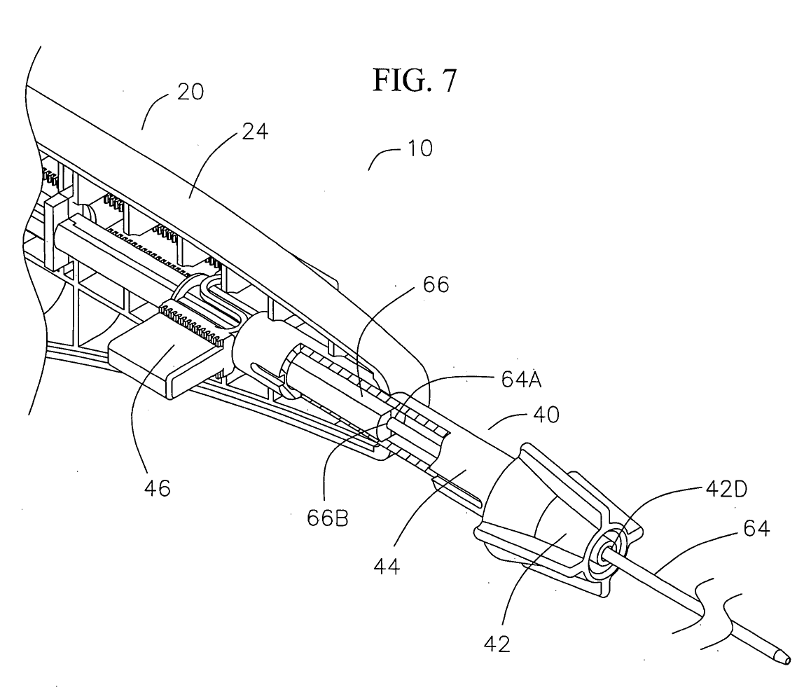

FIG. 7 is a partially sectioned perspective view of the fine needle aspiration instrument 10 with the right handle portion 22 removed wherein the shaft length adjuster assembly 40 is engaged with the left handle portion 24 of the handle assembly 20. The shaft length adjuster assembly 40 is partially advanced and the shaft adjuster tube 44 is partially sectioned to illustrate the interface of a proximal end 64A of the flex sheath 64 through a sheath recess 66B in the band guide 66 as the flex sheath 64 passes through a clearance hole 42D of the scope mount 42. Particular to this view, the spring lock button 46 moves in conjunction with the shaft adjuster tube 44 of the shaft length adjuster assembly 40 in relation to the handle assembly 20. The flex sheath 64 provides for flexible support of internal components, namely the grater tube 54 and cover tube 58 while preventing damage through torturous paths. The flex sheath 64 can be manufactured of medical grade coiled wire or provided in a form similar to a molded or extruded plastic tube.

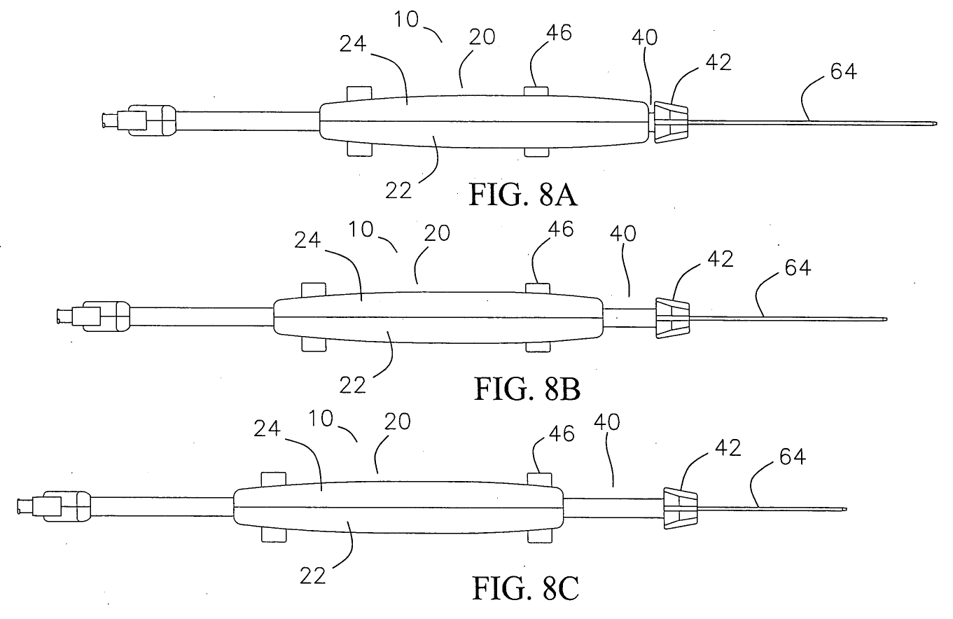

FIGS. 8A through 8C are top perspective views of the fine needle aspiration instrument 10 relative to a fixed location of the scope mount 42 where it attaches to the proximal end of an echoendoscope 100 (best shown in FIG. 2) and the related flex sheath 64 position. FIG. 8A shows the spring lock button 46 in its fully back position and the subsequent retracted position of the shaft length adjuster assembly 40 (forward position of the handle assembly 20) providing the longest length of the flex sheath 64. FIG. 8B shows the spring lock button 46 approximately halfway forward which provides an intermediate position of the shaft length adjuster assembly 40 (intermediate rearward position of the handle assembly 20) and the related partially retracted length of the flex sheath 64. FIG. 8C shows the spring lock button 46 in its fully forward position and the subsequent extended position of the shaft length adjuster assembly 40 (fully rearward position of the handle assembly 20) providing the shortest length of the flex sheath 64.

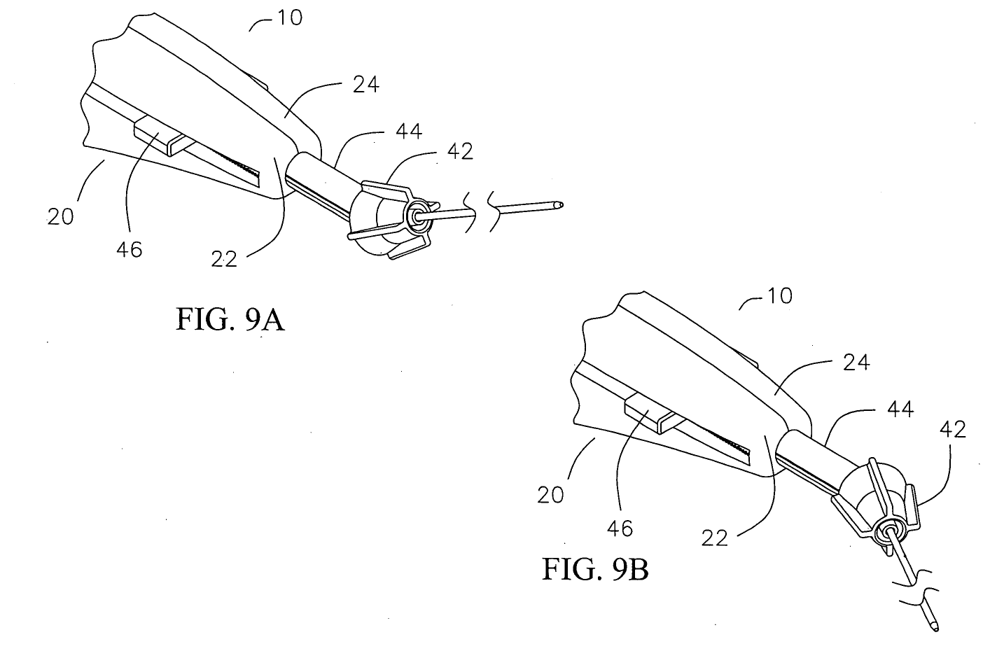

FIGS. 9A and 9B are partial perspective views of the fine needle aspiration instrument 10 illustrating the universal joint of the scope mount 42 and the shaft adjuster tube 44. FIG. 9A depicts the scope mount 42 fully flexed in an upward position in relation to the stationary shaft adjuster tube 44 and the handle assembly 20. FIG. 9B depicts the scope mount 42 fully flexed to the right in relation to the stationary shaft adjuster tube 44 and the handle assembly 20.

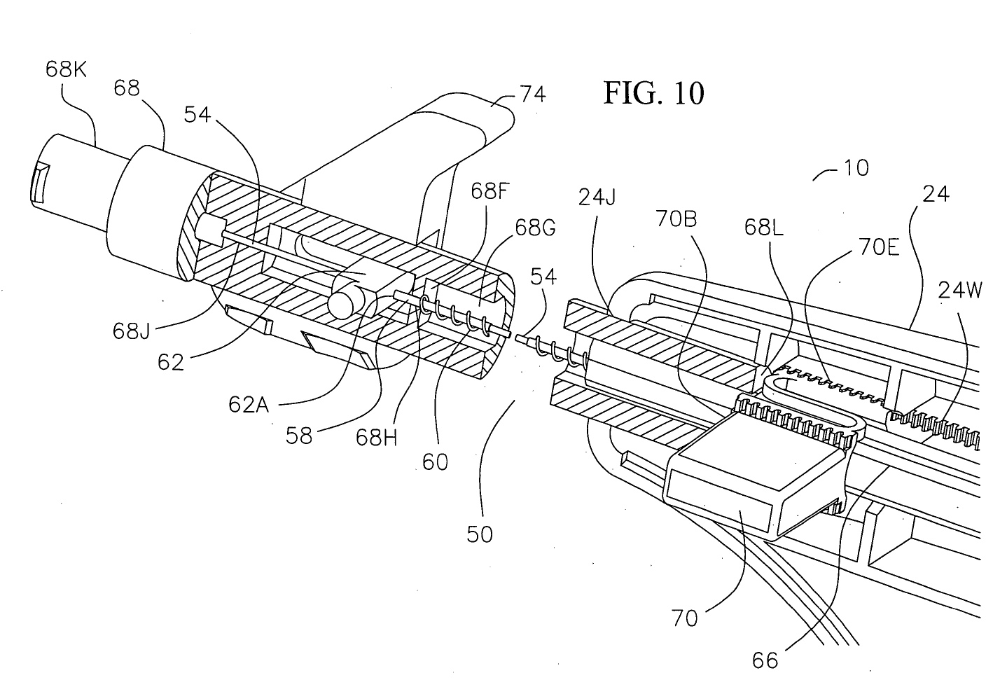

FIG. 10 is a close up perspective view of the fine needle aspiration instrument 10 in which the right handle portion 22 is removed and the needle control assembly 50 is detailed. Locking teeth 70E of spring lock button 70 are engaged with locking teeth 24W of the left handle portion 24 preventing the forward motion of the needle control tube 68 as a stop face 68L contacts a contact surface 70B of the spring lock button 70. The needle control tube 68 is constructed of an injection molded plastic and fits within a needle control shaft clearance hole 22J and 24J of the right handle portion 22 and left handle portion 24, respectively. The needle spring 60 is shown seated in a spring pocket 68G against a spring stop 68F. The cover tube 58 passes through the cover tube clearance 68H of the needle control tube 68 and into a cover tube hole 62A of the cam follower 62 where it is fixedly attached via press fit, adhesive, or other means. The cam follower 62 is an injection molded plastic part designed to fit within the needle control tube 68 and engage with the right cam half 72 and left cam half 74. The grater tube 54 passes through the cover tube 58 and the cam follower 62 where it comes to seat within a needle mount 68J of the needle control tube 68 distal to the female luer 68K, to which a vacuum source, such as an evacuated syringe (not shown), can be attached. The seated needle spring 60 provides a positive preload against the needle control tube 68.

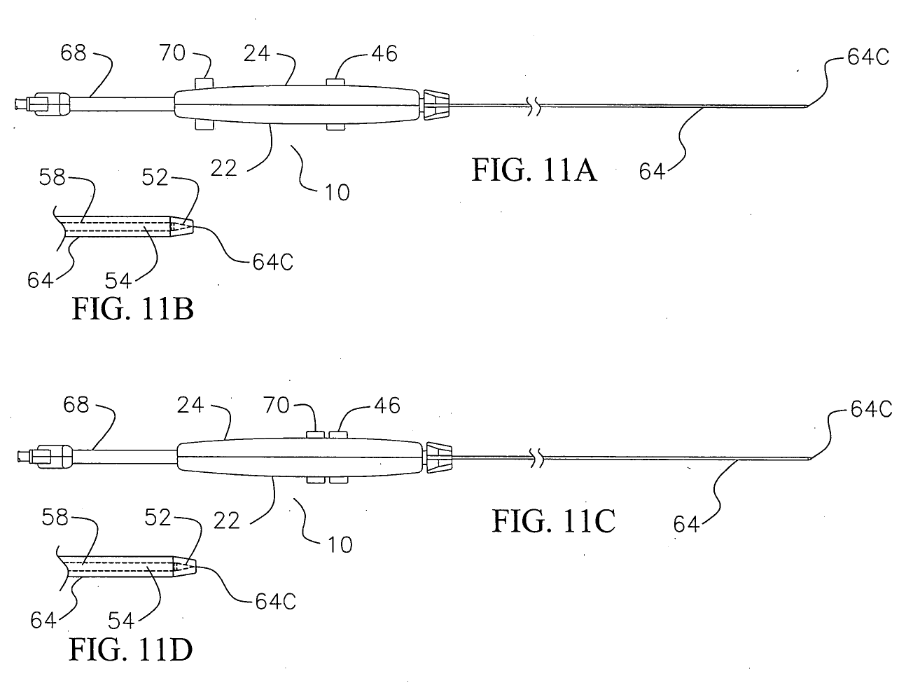

FIG. 11A is a top perspective view the fine needle aspiration instrument 10 showing the spring lock button 70 in a fully rearward position to prevent any forward motion of the needle control tube 68 along with attached grater tube 54 (not shown) such that the cover tube 58, grater tube 54, and needle tip 52 do not extend beyond the distal opening 64C of the flex sheath 64 as shown in FIG. 11B.

FIG. 11B is an enlarged schematic view of the distal end of FIG. 11A showing the cover tube 58 and needle tip 52 outlined inside of the flex sheath 64 of the fine needle aspiration instrument 10.

FIG. 11C is a top perspective view of the fine needle aspiration instrument 10 showing the spring lock button 70 in fully advanced position but with the needle control tube 68 along with attached grater tube 54 (not shown) remaining in the fully retracted state such that the cover tube 58, grater tube 54, and needle tip 52 do not extend beyond the distal opening 64C of the flex sheath 64.

FIG. 11D is an enlarged schematic view of the distal end of FIG. 11B showing the cover tube 58 and needle tip 52 outlined inside of the flex sheath 64 of the fine needle aspiration instrument 10.

FIG. 11E is a top perspective view the fine needle aspiration instrument 10 showing the spring lock button 70 in partially advanced position and the needle control tube 68 along with attached grater tube 54 (not shown) partially advanced such that the cover tube 58, grater tube 54, and needle tip 52 partially extend beyond the distal opening 64C of the flex sheath 64.

FIG. 11F is an enlarged schematic view of the distal end of FIG. 11E showing the cover tube 58 and needle tip 52 partially extended beyond the distal opening 64C of the flex sheath 64 of the fine needle aspiration instrument 10.

FIG. 11G is a top perspective view of the fine needle aspiration instrument 10 showing the spring lock button 70 in fully advanced and the needle control tube 68 along with attached grater tube 54 (not shown) fully advanced such that the cover tube 58, grater tube 54, and needle tip 52 fully extend beyond the distal opening 64C of the flex sheath 64.

FIG. 11H is an enlarged schematic view of the distal end of FIG. 11G showing the cover tube 58 and needle tip 52 fully extended beyond the distal opening 64C of the flex sheath 64 of the fine needle aspiration instrument 10.

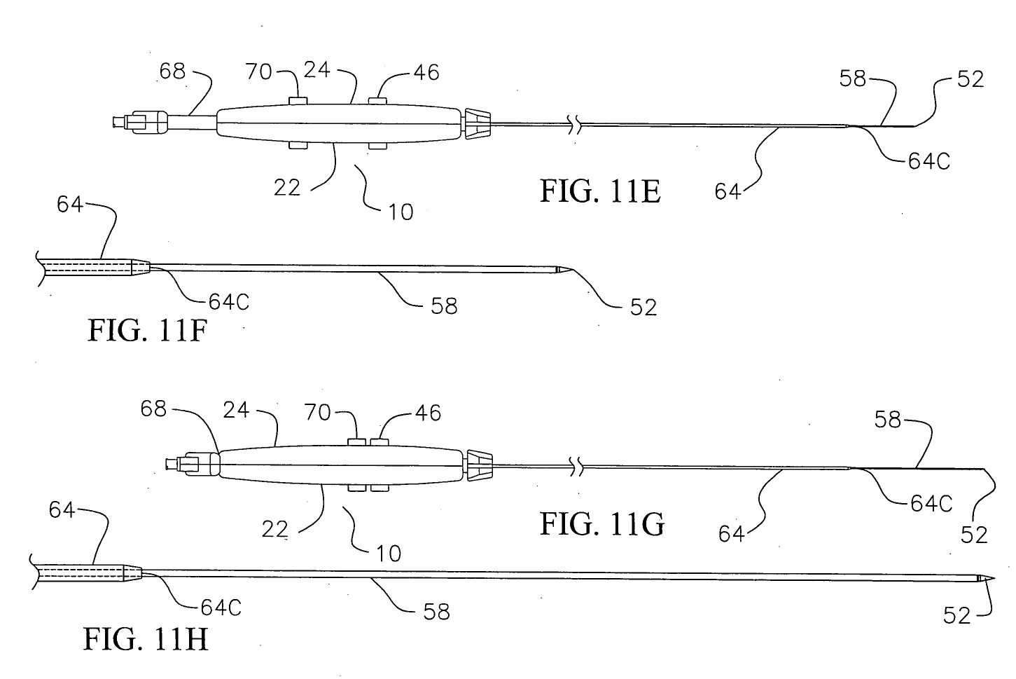

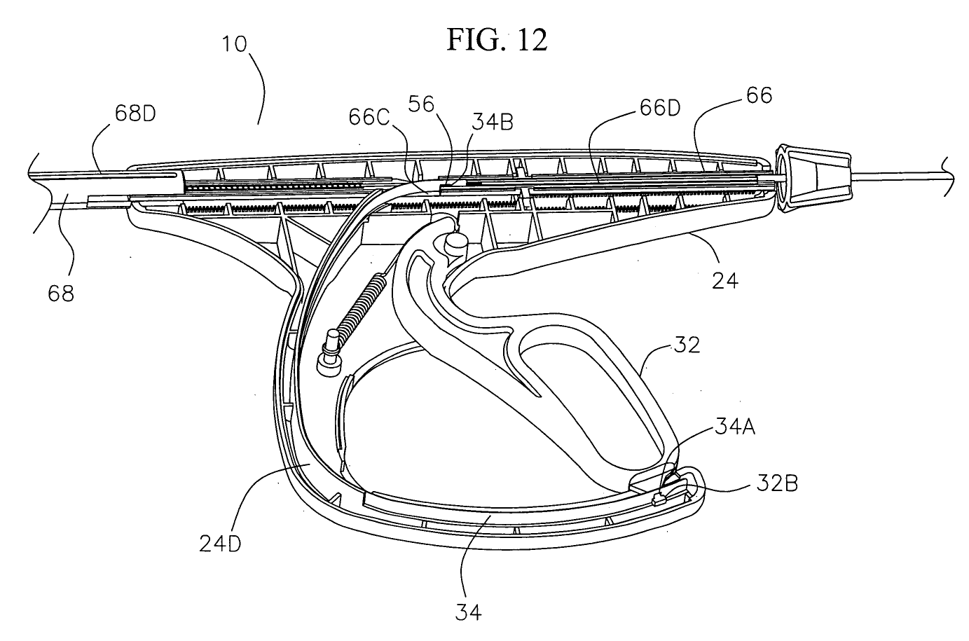

FIG. 12 is a perspective view of the fine needle aspiration instrument 10 in which the right handle portion 22, spring lock button 46, and spring lock button 70 are removed. Best illustrated in this view, the tab 32B of the lever 32 is engaged within the lever tab opening 34A of the band drive 34. The band drive 34 continues along the band drive wall 22D (not shown) and 24D of the right handle portion 22 (not shown) and the left handle portion 24, respectively, and through the band lead-in 66C where a drive tang opening 34B engages the drive tang 56. The band drive 34 nests within a band track 66D of the band guide 66, which prevents the lateral and vertical motion of the band drive 34 and disengagement from the drive tang 56. Also evident is an anti-rotation slot 68D of the needle control tube 68 which mates with a needle control anti-rotation nub 22H and 24H of the right handle portion 22 (not shown) and left handle 24 portion of FIGS. 4A and 4B.

FIGS. 13A and 13B are close-up exploded perspective views of selected components of the fine needle aspiration instrument 10 from FIG. 12. FIG. 13A is a fully exploded view illustrating the cover tube 58, an external grater tube 54 with an attached drive tang 56 in relation to the drive tang opening 58B, and band drive 34 with its drive tang opening 34B aligned with the drive tang 56. FIG. 13B is a partially exploded view illustrating the cover tube 58, with an internally assembled grater tube 54 with an attached drive tang 56 in relation to the drive tang opening 58B, and band drive 34 with its drive tang opening 34B aligned with the drive tang 56. FIGS. 13A and 13B best illustrates that the needle assembly of the present invention consists of two coaxial tubes, one within the other, or simply a double tube needle assembly.

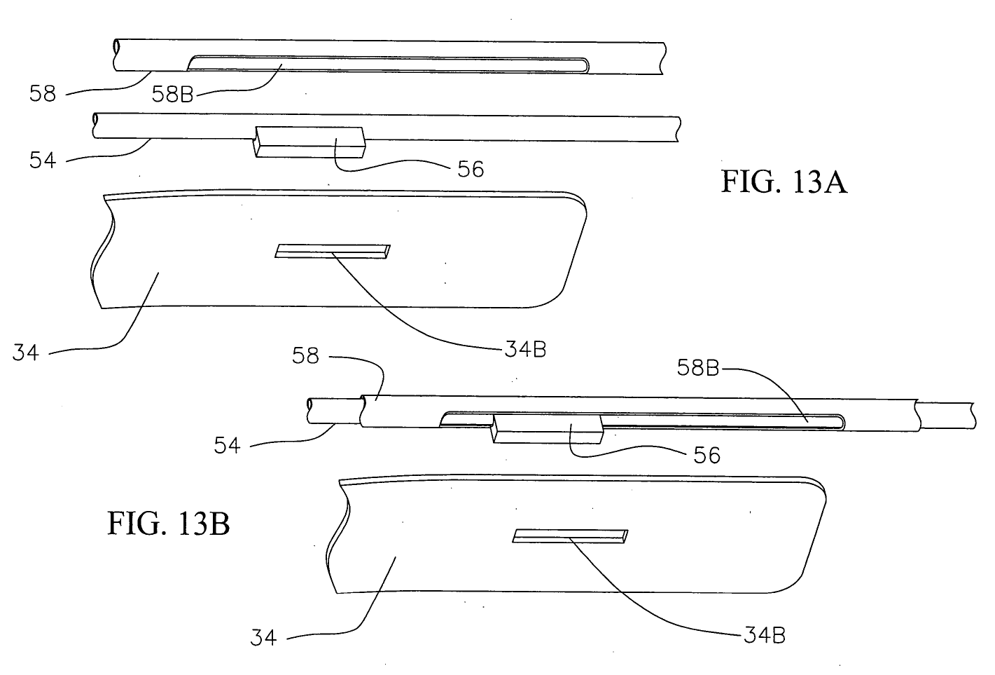

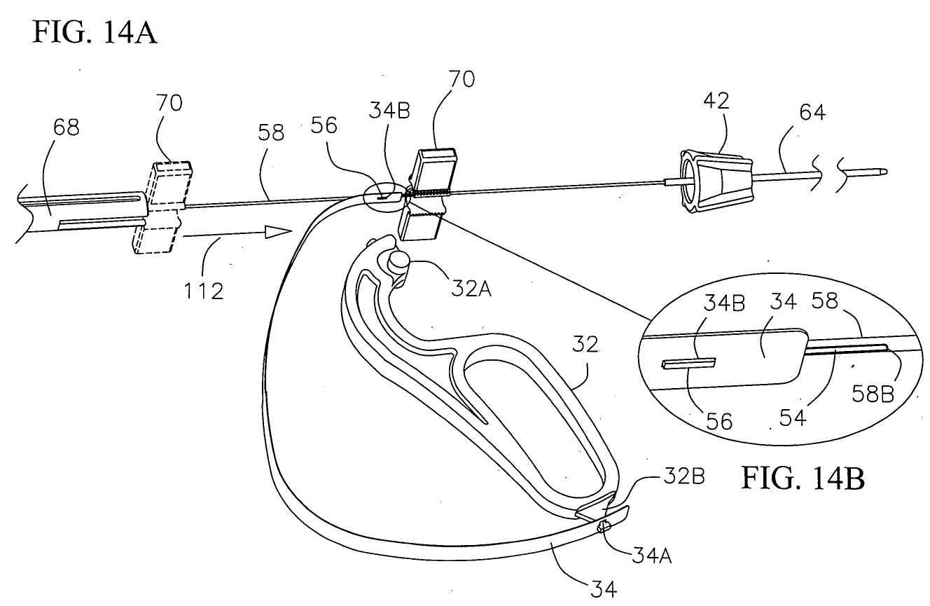

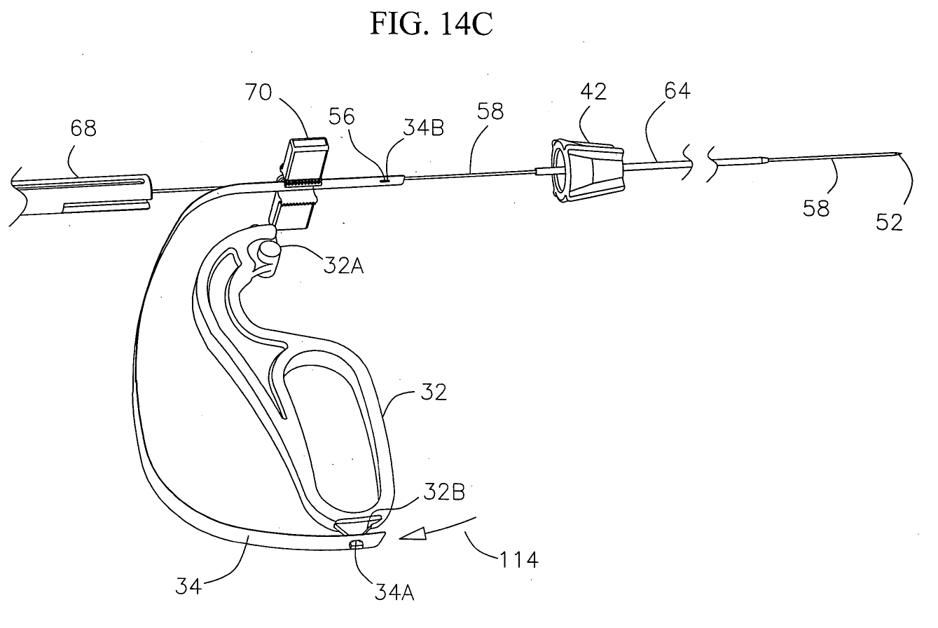

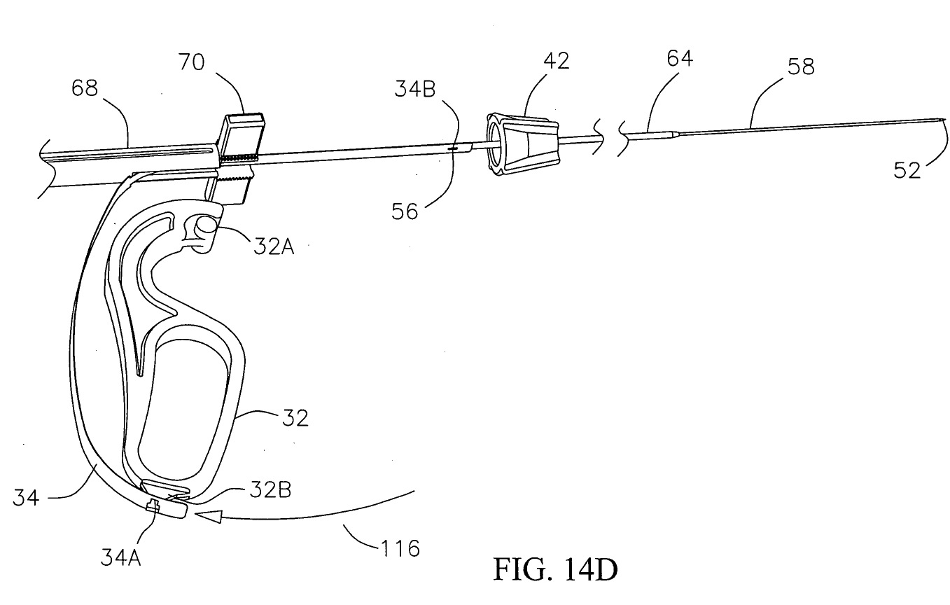

FIG. 14A is a perspective view of fine needle aspiration instrument 10 from FIG. 12 with various components removed for clarity. With the pivot 32A of the lever 32 stationary and the lever rotated fully forward, the tab 32B of the lever 32 is engaged with the lever tab opening 34A of the band drive 34. The drive tang opening 34B encompasses the drive tang 56 and holds the grater tube 54 via the drive tang 56 along with the cover tube 58 in a fully retracted state within the flex sheath 64. The needle control tube 68 attached to the proximal grater tube 54A is shown in its fully back position. The spring lock button 70 is shown fully forward, while the phantom lines shown and arrow 112 here indicate its most proximal position.

FIG. 14B is an enlarged perspective view of FIG. 14A detailing the connection of the drive tang opening 34B of the band drive 34 to the drive tang 56, which is fixedly attached to the grater tube 54 through the drive tang opening 58B of the cover tube 58.

FIG. 14C is a perspective view of fine needle aspiration instrument 10 from FIG. 12 with various components removed for clarity. With the pivot 32A of the lever 32. stationary and the lever partially rotated back shown by arrow 114, the tab 32B of the lever 32 is engaged with the lever tab opening 34A of the band drive 34. The drive tang opening 34B encompasses the drive tang 56 and advances the grater tube 54 (not shown) via the drive tang 56 along with the cover tube 58 partially outside the flex sheath 64. The needle control tube 68 is shown partially advanced, while the spring lock button 70 is shown fully forward.

FIG. 14D is a perspective view of the fine needle aspiration instrument 10 from FIG. 12 with various components removed for clarity. With the pivot 32A of the lever 32 stationary and the lever rotated fully back shown by arrow 116, the tab 32B of the lever 32 is engaged with the lever tab opening 34A of the band drive 34. The drive tang opening 34B encompasses the drive tang 56 and advances the grater tube 54 (not shown) via the drive tang 56 along with the cover tube 58 completely outside the flex sheath 64. Both the needle control tube 68 and the spring lock button 70 are shown fully forward.

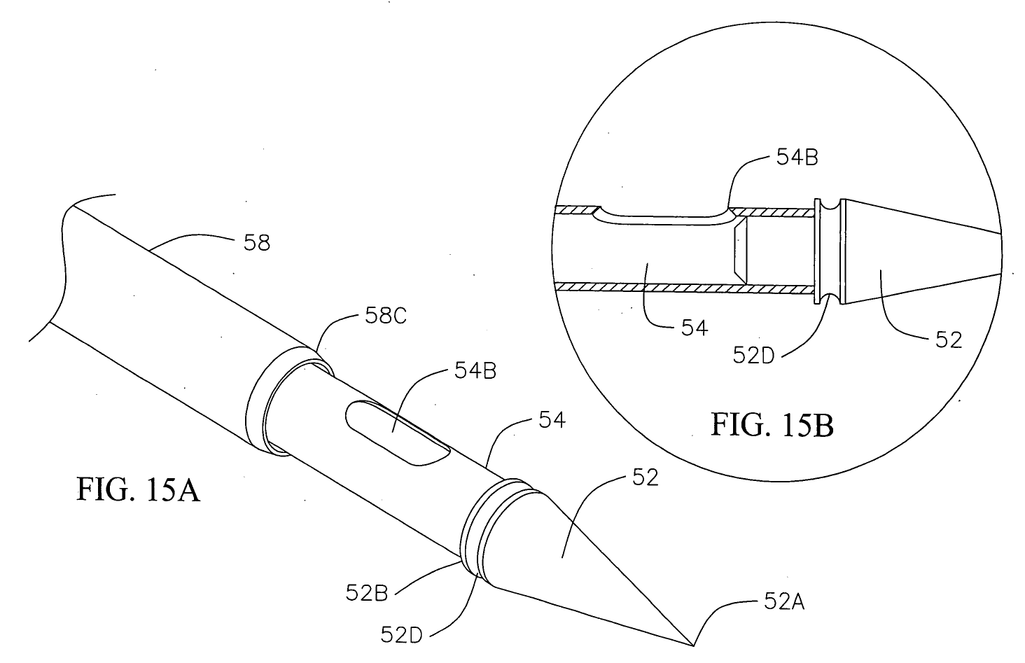

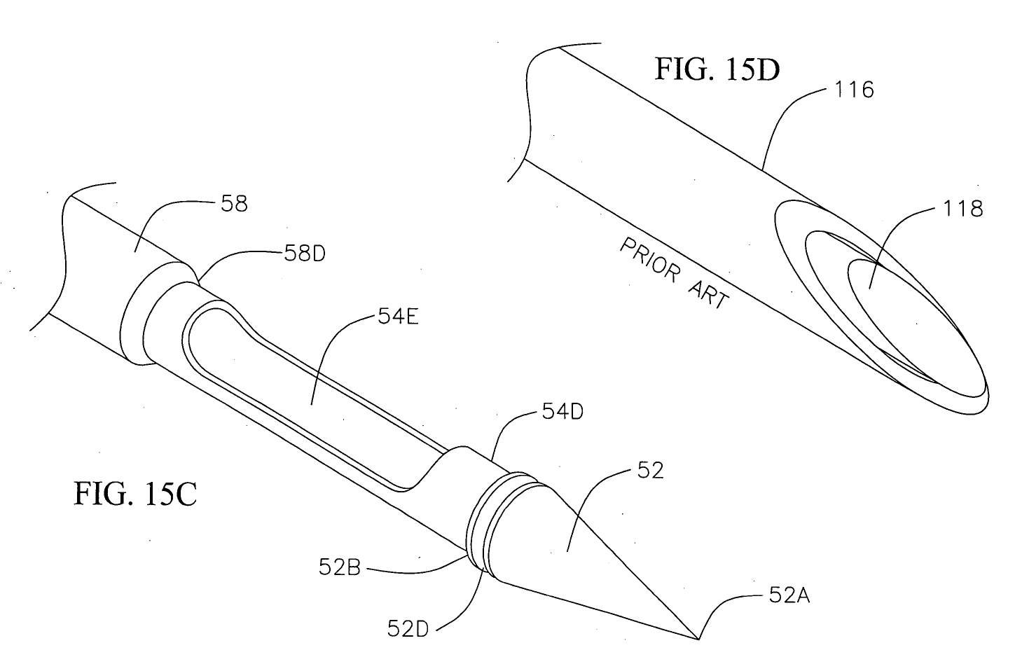

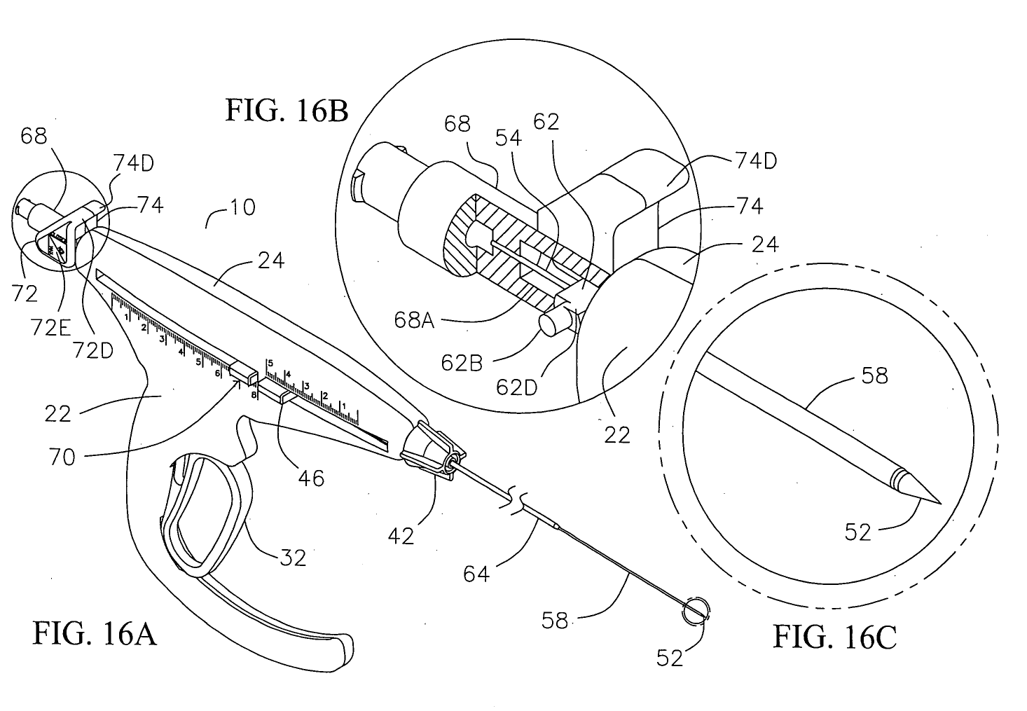

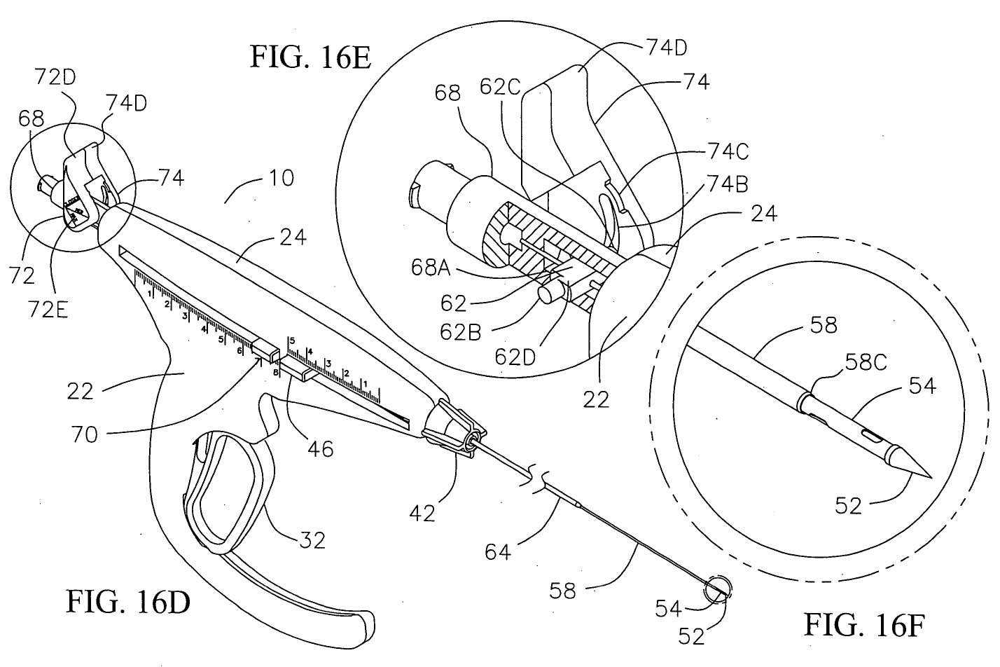

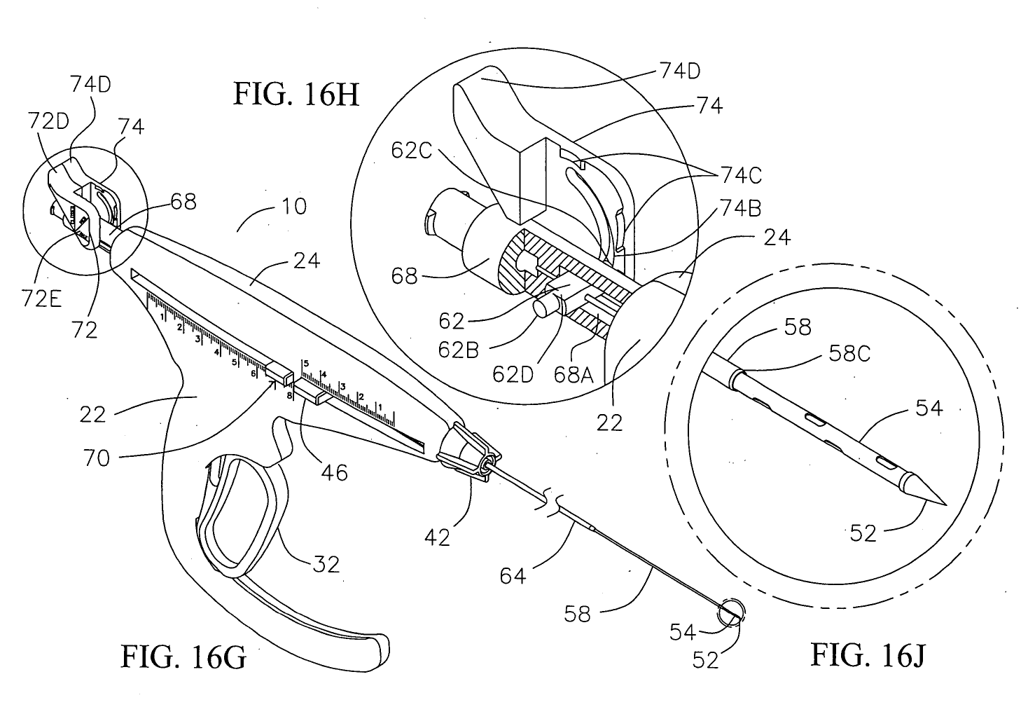

FIG. 15A is an enlarged perspective view of the cover tube 58, an exposed grater tube 54 and needle tip 52 of FIG. 14D. The grater tube 54 is exposed due to functions expressed in FIGS. 16A through 16J. The grater tube 54 and its attached needle tip 52 are shown with the distal chamfer 58C of cover tube 58 partially retracted back to expose the most distal sharpened hole 54B. The shoulder 52B of needle tip 52 is shown here with a circumferential groove or recess feature 52D to enhance the its potential to reflect back sound waves emitted from the echoendoscope thereby improving its ability to be imaged (i.e., its echogenicity).

FIG. 15B is an enlarged section view of FIG. 15A highlighting the edges of tissue scraping aperture in the form of a sharpened hole 54B of grater tube 54. When vacuum is applied to the lumen of grater tube 54 and its sharpened openings are exposed to tissue, the tissue is drawn into the sharpened hole or holes 54B. Movement or oscillation of the grater tube relative to the vacuum held tissue causes tissue fragments to be cut away from the targeted tissue at the tissue harvest site. As used herein, oscillation means moving the grater tube at least once in one direction, preferably moving the grater tube back and forth multiple times. Tissue fragments drawn into the grater tube 54 under these vacuum and oscillation harvest techniques are subsequently isolated by advancing the cover tube 58 (see FIG. 15A) back over the grater tube holes 54B.

While the sharpened holes provide an effective tissue-scraping aperture for harvesting tissue, other arrangements may also be used such as grating projections or the like.

FIG. 15C provides a perspective view of the distal tip of an alternative embodiment 54D to the aspiration needle shown in 15A with a larger single hole 54E and sharpened distal cover tube 58D for core biopsy. This embodiment permits a potentially larger portion of targeted tissue to be drawn by vacuum into the grater tube 54D and to be held in place. Advancing the sharpened cover tube 58D back over this hole and its contained tissue, cuts the sample tissue away from the targeted site and captures the specimen within the grater tube 54D.

FIG. 15D shows the distal end of a standard fine needle aspiration needle 116 with its indwelling beveled wire stylet 118 in accordance with the prior art. This traditional style of needle could also be incorporated into this invention to provide improved ergonomics, while permitting use of the traditional style of needle that physicians are currently trained to use.

FIG.16A is a perspective view of the fine needle aspiration instrument 10 wherein the spring button 70 and needle control tube 68 are fully forward and the lever 32 is fully back. A finger tab 72D and 74D of the right cam half 72 and mated left cam half 74, respectively, are oriented forward in their fully closed position as evidenced by a cover tube indicator 72E. The cover tube 58, its fully housed grater tube 54, and the needle tip 52 are fully advanced past the flex sheath 64.

FIG. 16B is an enlarged partially sectioned perspective view of FIG. 16A illustrating the position of the left cam half 74 in its fully forward position along with the mating cam follower 62. The cam follower 62 rests within a cam follower track 68A within the needle control tube 68 and a pair of anti-rotation flats 62D prevent the cam follower 62 from becoming wrongly oriented within the assembly. A right cam slot post 62B and left cam slot post 62C (not shown) engage with a cam slot 72B and cam slot 74B (not shown) of the right cam half 72 and mated left cam half 74, respectively (best shown in FIGS. 16E and 16H). The right cam half 72 and mated left cam half 74 are each held in position by a series of three detents 72C and 74C, respectively (best shown in FIGS. 16E and 16H), which are contoured to mate with the outer surface of the needle control tube 68. This particular positioning of the right cam half 72 and mated left cam half 74 prevents exposure of the grater tube 54 beyond the confines of the cover tube 58 as shown if FIG. 16C.

FIG. 16C is an enlarged perspective view of the fully advanced cover tube 58 and needle tip 52 of FIG. 16A.

FIG. 16D is a perspective view of the fine needle aspiration instrument 10 wherein the spring lock button 70 and needle control tube 68 are fully forward and the lever 32 is fully back. A finger tab 72D and 74D of the right cam half 72 and mated left cam half 74, respectively, are oriented upward and in their mid position as evidenced by a cover tube indicator 72E and partially retracts cover tube 58, leaving a partially exposed grater tube 54, and the needle tip 52 which are fully advanced past the flex sheath 64.

FIG. 16E is an enlarged partially sectioned perspective view of FIG. 16D illustrating the position of the left cam half 74 in upward orientation with the mating cam follower 62. The cam follower 62 rests within a cam follower track 68A within the needle control tube 68 and a pair of anti-rotation flats 62D prevent the cam follower 62 from becoming wrongly oriented within the assembly. A right cam slot post 62B and left cam slot post 62C engage with a cam slot 72B (not shown) and cam slot 74B of the right cam half 72 and mated left cam half 74, respectively. This particular positioning of the right cam half 72 and mated left cam half 74 partially retracts the cover tube 58 and exposes the grater tube 54 beyond the distal chamfer 58C of the cover tube 58 as shown if FIG. 16F.

FIG. 16F is an enlarged perspective view of the partially retracted cover tube 58 and partially exposed grater tube 54 and needle tip 52 of FIG. 16D.

FIG. 16G is a perspective view of the fine needle aspiration instrument 10 wherein the spring lock button 70 and needle control tube 68 are fully forward and the lever 32 is fully back. A finger tab 72D and 74D of the right cam half 72 and mated left cam half 74, respectively, are oriented fully back and in their max position as evidenced by a cover tube indicator 72E, and fully retracts the cover tube 58, and fully exposes the grater tube 54, and the needle tip 54, all of which are fully advanced past the flex sheath 64.

FIG. 16H is an enlarged partially sectioned perspective view of FIG. 16G illustrating the position of the left cam half 74 in a fully back orientation position with the mating cam follower 62. The cam follower 62 rests within a cam follower track 68A within the needle control tube 68 and a pair of anti-rotation flats 62D prevent the cam follower 62 from becoming wrongly oriented within the assembly. A right cam slot post 62B and left cam slot post 62C engage with a cam slot 72B (not shown) and cam slot 74B of the right cam half 72 and mated left cam half 74, respectively. This particular positioning of the right cam half 72 and mated left cam half 74 fully retracts the cover tube 58 thus fully exposing the grater tube 54 beyond the distal chamfer 58C of the cover tube 58 as shown if FIG. 16J.

FIG. 16J is an enlarged perspective view of the fully retracted cover tube 58 and fully exposed grater tube 54 and needle tip 52 of FIG. 16G.

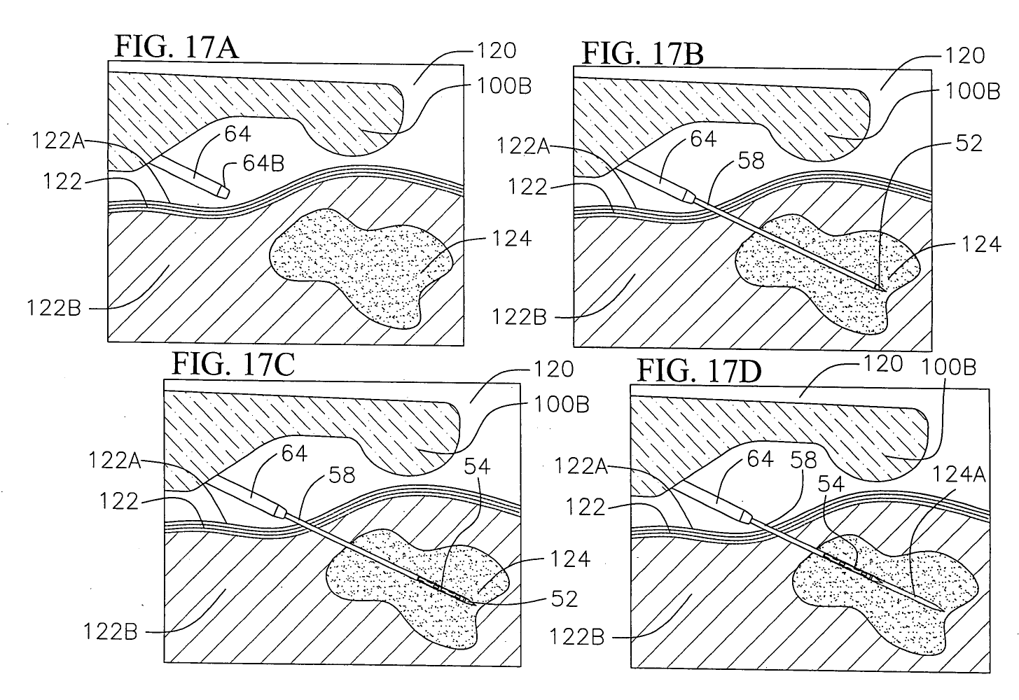

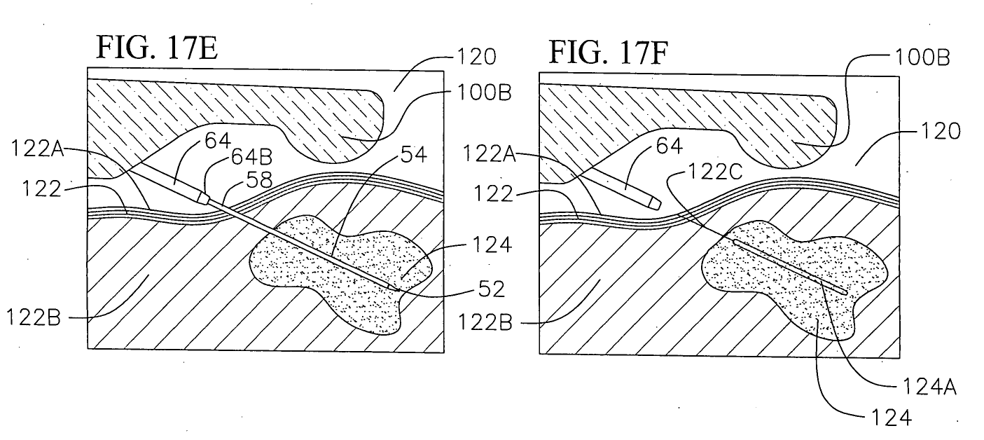

FIG. 17A is a schematic representation of the distal end of an echoendoscope 100B within the lumen of a hollow tissue space 120 and positioned over solid tissue structure 122 with a tissue surface lining 122A and underlying solid tissue 122B. An imaged targeted tissue site or lesion 124 is contained within tissue 122. A flex sheath 64 with its chamfered distal end 64B protrudes from distal end of an echoendoscope 100.

FIG. 17B is a schematic representation of the distal end of an echoendoscope 100B with a protruding distal end of a flexible sheath 68 positioned over a tissue segment 122 containing an imaged lesion 124 and with the double tube needle completely covered with a non-retracted cover tube 58 penetrating through the overlying tissue 122A and 122B and deeply into the lesion 124.

FIG. 17C is a schematic representation of the distal end of an echoendoscope 100B with a protruding distal end of a flexible sheath 68 positioned over a tissue segment 122 containing an imaged lesion 124 and a double tube needle penetrating through the overlying tissue deeply into the lesion, but now the cover tube 58 is shown fully retracted back to expose the sharpened holes of the grater tube 54, through which a vacuum is now applied.

FIG. 17D is a schematic representation of the distal end of an echoendoscope 100B with a protruding distal end of a flexible sheath 68 positioned over a tissue segment 122 containing an imaged lesion 124 and the double tube needle with the cover tube 58 fully retracted exposing the sharpened holes of the vacuum augmented grater tube 54 now pulled partially back up through the lesion 124 but not into the overlying tissue 122B.

FIG. 17E is a schematic representation of the distal end of an echoendoscope 100B with a protruding distal end of a flexible sheath 68 positioned over a tissue segment 122 containing an imaged lesion 124 at completion of the oscillation harvesting technique and the double tube needle now with the cover tube 58 fully forward covering the sharpened holes of the grater tube 54 (not shown) and protecting the harvested specimen from contamination. At this point, the vacuum is typically shut off.

FIG. 17F is a schematic representation of the distal end of an echoendoscope 100B with a protruding distal end of a flexible sheath 68 positioned over a tissue segment 122 containing an imaged lesion 124 at completion of the fine needle aspiration harvesting technique with the double tube needle containing the protected specimen now back completely within the sheath and also showing a representative needle track 122C through the overlying tissue and the specimen harvest site 124A in the imaged lesion.

1. An instrument for harvesting a tissue sample in a medical procedure comprising: an elongated hollow needle, a penetrating tip on a distal end of the needle, one or more tissue scraping apertures in a side wall of the elongated hollow needle, a cover tube coaxially disposed on the elongated hollow needle, slidably movable on the elongated hollow needle from a first position exposing the one or more tissue scraping apertures to a second position covering the one or more tissue scraping apertures.

2. The instrument of claim 1 in which the tissue scraping apertures comprise holes formed in the sidewall of the needle, the holes having tapering sidewalls and characterized by a larger diameter on an inside surface of the sidewall than on an outside surface.

3. The instrument of claim 1 comprising a plurality of apertures radially spaced around the sidewall of the needle.

4. The instrument of claim 1 comprising a plurality of apertures axially spaced along the sidewall of the needle.

5. The instrument of claim 1 comprising a sheath surrounding the cover tube and the elongated hollow needle, the cover tube and needle being movable in the sheath from a withdrawn position to an extended position.

6. The instrument of claim 1 in which the sheath is attached to a needle shaft length adjustment tube received in the body, the shaft length adjustment tube being movable to change the length of the needle and the cover tube that extend beyond the end of the sheath when the needle is extended.

7. The instrument of claim 1 comprising a handle having a body and a lever pivotally attached to the body, the elongated hollow needle coupled to the lever for reciprocating the needle relative to the tissue to be harvested.

8. The instrument of claim 1 comprising a spring coupled between the lever an the body, biasing the lever to a position in which the needle is retracted relative to the body.

9. The instrument of claim 1 in which the elongated hollow needle is coupled to the handle by an elongated thin band.

10. The instrument of claim 1 in which the elongated thin band is coupled to a distal end of the lever, remote from the pivot.

11. The instrument of claim 1 comprising an elongated track in the body and in which the needle and the elongated thin band are slidably supported by the track.

12. The instrument of claim 1 in which the body includes an extension and the thin band is enclosed in the extension, and the distal end of the lever engages the band through a slot in the extension.

13. The instrument of claim 1 comprising a handle having a body and a lever pivotally attached to the body, the elongated hollow needle coupled to the lever for reciprocating the needle relative to the tissue to be harvested.

14. The instrument of claim 1 comprising an adjustable sleeve, attached to the body, and coupled to the sheath for adjusting the amount by which the needle protrudes from the sheath when the needle is in the extended position.

15. The instrument of claim 1 comprising a lock attached to the adjustable sleeve.

16. The instrument of claim 1 in which the lock comprises a toothed rack, and a movable locking member selectively engaging the toothed rack.

17. The instrument of claim 1 in which the movable locking member comprises an actuator extending outside of the body.

18. The instrument of claim 1 in which the lock comprises a releasable lock coupled to the body.

19. The instrument of claim 1 in which the sheath is pivotally attached to the body.

20. The instrument of claim 1 in which the pivotal attachment includes a plurality of detents.

21. The instrument of claim 1 comprising a reciprocating plunger, movable from a retracted position to an extended position and attached to the handle and coupled to the needle and the cover tube for simultaneously extending the needle and the cover tube from the sheath when the plunger is in the extended position.

22. The instrument of claim 1 comprising an adjustable stop coupled to the plunger for controlling the distance by which the needle and cover are extended from the sheath in the extended position.

23. The instrument of claim 1 in which the adjustable stop comprises a toothed rack, and a movable locking member selectively engaging the toothed rack.

24. The instrument of claim 1 comprising a lever attached to the plunger and the cover tube for retracting the cover tube relative to the hollow elongated needle.

25. The instrument of claim 1 in which the lever is pivotally attached to the plunger and includes a boss projecting through a sidewall of the plunger into engagement with a proximal end of the needle.

26. The instrument of claim 1 in which the boss rides in a cam slot in the lever.

27. The instrument of claim 1 comprising a spring biasing the plunger to the retracted position.

28. The instrument of claim 1 comprising a vacuum source coupled to the elongated hollow needle for drawing a tissue sample into the one or more apertures.

29. A method of harvesting a tissue sample comprising:
imaging the location of the tissue to be sampled;
providing a thin hollow grater tube having a tissue penetrating tip;
providing a smooth cover tube selectively covering the grater tube;
moving the cover tube into a position covering the grater tube;
inserting the cover tube and grater tube into the tissue to be sampled;
retracting the cover tube to expose the grater tube;
oscillating the grater tube to collect a portion of the tissue to be sampled;
advancing the cover tube to cover the grater tube;
withdrawing the cover tube and grater tube from the tissue to be sampled; and
extracting the collected portion of the tissue to be sampled from the grater tube.

30. The method of claim 29 comprising maintaining a vacuum in the grater tube during the oscillation step.

31. The method of claim 29 in which the imaging step comprises ultrasonically imaging the location of the tissue to be sampled.

32. The method of claim 29 in which the inserting step comprises inserting through an endoscope.

33. The method of claim 29 comprising attaching a manually operable lever to the grater tube and in which the oscillating step comprises moving the lever.

34. The method of claim 32 in which said cover tube and said grating tube are coaxially disposed in a sheath, and in which said inserting step comprises inserting the sheath through an endoscope port.

35. The method of claim 32 in which said withdrawing step comprises withdrawing the cover tube and grating tube from the endoscope.

36. A spring lock for a medical instrument having two opposed instrument halves comprising a fixed toothed rack on at least one instrument half; at least one lock button extending through one of the instrument halves, a movable toothed rack segment coupled to the button and selectively engaging the toothed rack on the instrument half and a spring urging the fixed rack and movable rack segments into engagement.

37. The spring lock of claim 36 in which the button and the spring are formed integrally.

38. The spring lock of claim 36 comprising a second button attached to the spring.

39. The spring lock of claim 36 comprising a second button attached to the spring and a second movable rack attached to the second button.

40. An echogenically imagable surgical instrument comprising an instrument tip and a groove formed in the instrument tip.

41. The surgical instrument of claim 40 comprising a needle in which the tip is a solid tip.

42. The surgical instrument of claim 40 comprising a grating tube in which the tip is a solid tip.

43. A medical instrument having a handle, a lever attached to the handle, and a longitudinally movable operating part attached to the handle comprising: a substantially rigid elongated flexible band connected between a distal end of the lever and the longitudinally movable operating part.

44. The medical instrument of claim 43 comprising a guide channel in the handle carrying a proximal end of the longitudinally movable operating part.

45. The medical instrument of claim 44 in which the flexible band is attached to the proximal end of the longitudinally movable operating part adjacent to the guide channel.
